(19) United States
(12) Patent Application Publication
(10) Pub. No.: US 2020/0085767 A1
(43) Pub. Date: Mar. 19, 2020

(54) Methods for Treating Hydrocephalus

(71) Applicant: Indian University Research and Technology Corporation, Indianapolis, IN (US)

(72) Inventor: Bonnie L. Blazer-Yost, Indianapolis, IN (US)

(21) Appl. No.: 16/470,699

(22) PCT Filed: Dec. 22, 2017
(86) PCT No.: PCT/US2017/068109

§ 371 Date: Jun. 18, 2019

Related U.S. Application Data
(60) Provisional application No. 62/438,174, filed on Dec. 22, 2016.

(51) Int. Cl. A61K 31/138

(52) U.S. Cl. CPC A61K 31/138

(57) ABSTRACT

Methods for inhibiting transepithelial ion transport, inhibiting hydrocephalic development, and for treating hydrocephalus are disclosed herein. The methods include administering a potassium channel inhibitor to the individual. In one particular embodiment, the individual is administered the calcium-activated potassium channel inhibitor, fluoxetine.

### CROSS-REFERENCE TO RELATED APPLICATIONS

This application claims priority to U.S. Provisional Application No. 62/438,174 filed on Dec. 22, 2016, which is hereby incorporated by reference in its entirety.

### BACKGROUND OF THE DISCLOSURE

The present disclosure relates generally to treating hydrocephalus. More particularly, the present disclosure relates to methods for inhibiting transepithelial ion transport, methods for inhibiting hydrocephalic development, and methods for treating hydrocephalus by administering inhibitors of potassium channels, and in particular, calcium-activated potassium channels such as fluoxetine.

Hydrocephalus is the buildup of fluid (cerebrospinal fluid-CSF) within the ventricular system of the brain, sometimes referred to as “water on the brain.” This is a serious condition that leads to neuronal death and long-term complications. CSF is produced by the choroid plexus (CP), which is an epithelial cell lined tuft of capillaries that project into the ventricles. The ventricle system is composed of four chambers, lateral ventricles (LV) within each of the cerebral hemispheres, the third ventricle connected to the lateral ventricles in the diencephalon (toward the base of the brain) and a fourth ventricle that is connected to the third ventricle by the cerebral aquaduct (of Sylvius). The CSF escapes from the fourth ventricle through three foramen (of Luschka and Magendie) into the subarachnoid space which surrounds the brain. CSF is reabsorbed into the venous blood by arachnid granulations attached to the superior sagittal sinus which lies just under the cranial skullcap.

The CSF volume in the brain can be increased as a consequence of either: a) production of excess CSF, b) blocking the flow of CSF (usually at the cerebral aquaduct) or c) insufficient reabsorption into the venous blood. The basic types of hydrocephalus are 1) communicating (there is no blockage of flow so fluid increase may be from over production or limited reabsorption) and non-communicating (there is blockage to flow of CSF out of the brain ventricles).

In infants and children who develop hydrocephalus, the cranial sutures have not yet fused, so the cranium enlarges as the ventricles enlarge. In adults with hydrocephalus, the fused nature of the adult cranium results in increased pressure and tissue damage.

Transient receptor potential cation channel subfamily V member 4 (TRPV4) is a member of the OSM9-like transient receptor potential channel (OTRPC) subfamily that in humans is encoded by the TRPV4 gene. TRPV4 protein is a Ca²⁺-permeable, nonselective cation channel that is thought to be involved in the regulation of systemic osmotic pressure. Particularly, TRPV4 is activated by changes in osmotic balance (hypotonicity) and mechanical stress (pressure) and, when activated, transports Ca²⁺ into cells. TRPV4 is expressed by the epithelial cells of the choroid plexus (CP) that is responsible for cerebrospinal fluid (CSF) generation. A number of TRPV4 agonists and antagonists have been identified including, for example, the antagonist Ruthenium Red (ruthenium oxychloride), the agonist 4aPDD (4a-Phorbol 12,13-didecanoate), the selective antagonist RN-1734 (2,4-Dichloro-N-isopropyl-N-(2-isopropylaminoethyl)benzenesulfonamide), the agonist GSK1016790A ((N-((1S)-1-{[4-(2S)-2-{[(2,4-Dichlorophenyl)sulfonyl]amino}-3-hydroxypropanoyl)-1-piperazinyl]carbonyl}-3-methylbutyl)-1-benzothiophene-2-carboxamide) and the antagonist HC-067047 (2-Methyl-1-[3-(4-morpholinyl)propyl]-5-phenyl-N-[3-(trifluoromethyl)phenyl]-1H-pyrrole-3-carboxamide).

Recently, the regulated ion transport, which is stimulated in response to TRPV4 agonists in a CP cell line, has been studied. When the native TRPV4 in the CP epithelia is stimulated, the channel allows calcium to enter the cell. The change in intracellular calcium could stimulate other calcium-activated ion channels. It has now been found that in response to TRPV4 agonists, there is a transepithelial ion movement that is consistent with either anion absorption or cation secretion across the CP epithelium. Since the ion transport dictates the secondary movement of water across epithelia, this would be followed by water movement and a build-up of excess CSF. Inhibition of calcium-activated potassium channels with fluoxetine inhibits TRPV4 agonist stimulated cation secretion into the CSF.

Accordingly, there exists a need to develop alternative treatments for inhibiting the TRPV4-stimulated ion transport, thereby reducing water movement and a build-up of excess CSF. It would further be advantageous if the treatments could be used to prevent the formation of hydrocephalus.

### BRIEF DESCRIPTION OF THE DISCLOSURE

The present disclosure relates generally to inhibiting transepithelial ion transport, inhibiting hydrocephalic development, and treating hydrocephalus. More particularly, the present disclosure relates to the use of inhibitors of potassium channels, such as calcium-activated potassium channels (e.g., fluoxetine) to inhibit the TRPV4-stimulated ion transport, thereby inhibiting the secondary movement of water across epithelia and build-up of excess CSF.

In one aspect, the present disclosure is directed to a method for inhibiting transepithelial ion transport in an individual in need thereof, the method comprising: administering a potassium channel inhibitor to the individual. In one particular embodiment, the potassium channel inhibitor is a calcium-activated potassium channel inhibitor.

In another aspect, the present disclosure is directed to a method for inhibiting hydrocephalic development in an individual in need thereof, the method comprising: administering a potassium channel inhibitor to the individual. In one particular embodiment, the potassium channel inhibitor is a calcium-activated potassium channel inhibitor.

In another aspect, the present disclosure is directed to a method for treating hydrocephalus in an individual in need thereof, the method comprising: administering a potassium channel inhibitor to the individual. In one particular embodiment, the potassium channel inhibitor is a calcium-activated potassium channel inhibitor.

In another aspect, the present disclosure is directed to a method for inhibiting fluid movement caused by transepithelial ion transport in an individual in need thereof, the method comprising: administering a potassium channel inhibitor to the individual. In one particular embodiment, the potassium channel inhibitor is a calcium-activated potassium channel inhibitor.

In accordance with the present disclosure, methods have been discovered that surprisingly allow for inhibiting transepithelial ion transport related to TRPV4 agonist stimulation. The present disclosure has a broad and significant impact, as it allows for treating hydrocephalus in subjects.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts the inhibitory effect of fluoxetine on transepithelial ion transport response brought on by the TRPV4 agonist, GSK1016790A, as discussed in Example 1.

FIG. 2 depicts measurement of the conductance change caused by pretreatment of fluoxetine, as discussed in Example 1.

FIG. 3 depicts the inhibitory effect of fluoxetine and norfluoxetine on transepithelial ion transport response brought on by the TRPV4 agonist, GSK1016790A, as discussed in Example 2.

FIG. 4 depicts measurement of the conductance change caused by pretreatment of fluoxetine and norfluoxetine, as discussed in Example 2.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Hydrocephalus

The term “hydrocephalus” refers to any hydrocephalus occurring at any point. For example, “hydrocephalus” can refer to any hydrocephalus occurring in the fetal or neonatal period including, but not limited to, Early Onset hydrocephalus (EOHC), Fetal Onset hydrocephalus, Congenital hydrocephalus, Obstructive hydrocephalus, Communicating hydrocephalus. Also included is any hydrocephalus resulting from structural defects in the brain, including but not limited to, ventricular defect; aqueductal stenosis; genetic defect; developmental defect; spina bifida; encepholocele; premature birth; cranial lesion; holoprosencephaly; dilatation of the lateral ventricles of the brain; internal hydrocephalus; functional impairment of the arachnoidal granulations (also called arachnoid granulations or Pacchioni's granulations); enlarged cerebral ventricles; and myelomeningocele, as well as any hydrocephalus occurring as a result of metabolic defects, including, but not limited to Dandy-Walker Syndrome; Walker-Wardburg syndrome, Meckel syndrome, Smith-Lemli-Opitz syndrome, chondrodystrophies, trisomy 13, trisomy 18, triploidy, congenital heart disease and cleft lip and/or palate; mutation to the L1 cell adhesion molecule; MASA; CRASH; communicating hydrocephaly; non-communicating hydrocephaly; increased intracranial pressure; normal pressure hydrocephaly; impaired cerebrospinal fluid (CSF) flow; impaired CSF reabsorption; excessive CSF production; congenital absence of arachnoid villi; and type II Arnold-Chiari malformation, and any hydrocephalous occurring as a result of brain injury, including but not limited to, surgical trauma; blunt force trauma; stroke; intraventricular hemorrhage; subarachnoid hemorrhage; traumatic brain injury; scarring and/or fibrosis of the subarachnoid space; and intra-ventricular matrix hemorrhages in a premature infant. Hydrocephalus includes hydrocephalus occurring as a result of pathological conditions, including, but not limited to, meningitis; encephalitis; benign tumor; cancerous tumor; cancer; neoplasm; papilloma of choroid plexus; brain atrophy; dementia; schizophrenia; brain parenchyma loss; colloid cyst; atresia; normal pressure hydrocephalus (NPH); and ependymitis. Hydrocephalus also includes any hydrocephalous occurring as a result of toxicities including, but not limited to, drug overdose; drug-drug interaction; poisoning; radiation; and idiopathic toxicity. And, also includes, any hydrocephalus occurring as a result brain inflammations, including, but not limited to, sepsis; allergy; and idiopathic inflammation.

Methods of Use

In accordance with the present disclosure, treatments have been discovered that surprisingly allow for treating diseases and disorders resulting from increased TRPV4-stimulated ion transport. Transepithelial ion transport can lead to water movement and build-up of excess CSF, thereby leading to the formation of hydrocelphalus.

Accordingly, in one aspect, the present disclosure is directed to a method for inhibiting transepithelial ion transport in an individual in need thereof. The method comprises: administering a potassium channel inhibitor to the individual. In one embodiment, the potassium channel inhibitor is a calcium-activated potassium channel inhibitor such as fluoxetine (Prozac). In another embodiment, the potassium channel inhibitor is norfluoxetine, which is an active metabolic product of fluoxetine.

In another aspect, the present disclosure is directed to a method for inhibiting hydrocephalic development in an individual in need thereof. The method comprises: administering a potassium channel inhibitor to the individual. In one embodiment, the potassium channel inhibitor is a calcium-activated potassium channel inhibitor such as fluoxetine. In another embodiment, the potassium channel inhibitor is norfluoxetine.

In another aspect, the present disclosure is directed to method for treating hydrocephalus in an individual in need thereof, the method comprising: administering a potassium channel inhibitor to the individual. In one embodiment, the potassium channel inhibitor is a calcium-activated potassium channel inhibitor such as fluoxetine. In another embodiment, the potassium channel inhibitor is norfluoxetine.

As used herein “an individual in need thereof” refers to an individual or subject susceptible to or at risk of a specified disease, disorder, or condition, particularly, hydrocephalus. Such individuals may include, but are not limited to, individuals susceptible to or at elevated risk of hydrocephalus due to any one or more of the causes as identified above. Further, individuals may be susceptible to or at elevated risk for hydrocephalus due to family history, age, environment, and/or lifestyle.

In one particular embodiment, the individual in need is an individual having a condition resulting in activation of TRPV4 such as a condition selected from increased intracranial pressure, decreased cerebral spinal fluid (CSF) osmolarity, presence of endogenous TRPV4 agonists such as inflammatory mediators (e.g., arachidonic acid metabolites or lysophosphatidic acid) that have escaped from the vasculature of the brain due to injury or inflammation. As used herein “transient receptor potential cation channel subfamily V member 4 agonist” or “transient receptor potential vanilloid 4 agonist” or “TRPV4 agonist” refers to any compound capable of activating or enhancing the biological activities of a TRPV4 channel receptor. Suitable agonists to TRPV4 channel receptors can be, for example, compounds included in the class of 3-oxohexahydro-1H-azepin, azepine and acyclic 1,3-diamine and derivatives of these compounds. Suitable TRPV4 agonists can be, for example, GSK1016790A (N-((1S)-1-{[4-(2S)-2-{[(2,4-dichlorophenyl)sulfonyl]amino}-3-hydroxypropanoyl)-1-piperazinyl]carbonyl}-3-methylbutyl)-1-benzothiophene-2-carboxamide); N-{1S)-1-[({(4R)-1-└(4-chlorophenyl)sulfonyl┘-3-oxohexahydro-1H-azepin-4-yl}amino)carbonyl┘-3-methylbutyl}-1-benzothiophene-2-carboxamide; N-(1S)-1-[({(4R)-1-[(4-fluorophenyl) sulfonyl]-3-oxohexahydro-1H-azepin-4-yl}amino)carbonyl]-3-methylbutyl}-1-benzothiophene-2-carboxamide; N-(1S)-1-[({(4R)-1-[(2-cyanophenyl)sulfonyl]-3-oxohexahydro-1H-azepin-4-yl}amino)carbonyl]-3-methylbutyl}-1-methyl-1H-indole-2-carboxamide; N-{(1S)-1-[({(4R)-1-┌(2-cyanophenyl)sulfonyl┐hexahydro-1H-azepin-4-yl}amino)carbonyl┐-3-methylbutyl}-1-methyl-1H-indole-2-carboxamide; N-{(1S)-1-[({3-└└(cyanophenyl)sulfonyl┘(methyl)amino┘propyl}amino)carbonyl┘-3-methylbutyl}-1-benzothiophene-2-carboxamide; and N-{(1S)-1-[({3-[[(2,4-dichlorophenyl)sulfonyl](methyl)amino]propyl}amino)carbonyl]-3-methylbutyl}-1-benzothiophene-2-carboxamide. Other suitable TRVP4 agonists are disclosed in US 2007/0259856 and International Patent Applications WO 2000/03687, WO 2001/095911, WO 2002/017924, and WO 2006/029209, which are incorporated by reference herein to the extent they are consistent herewith.

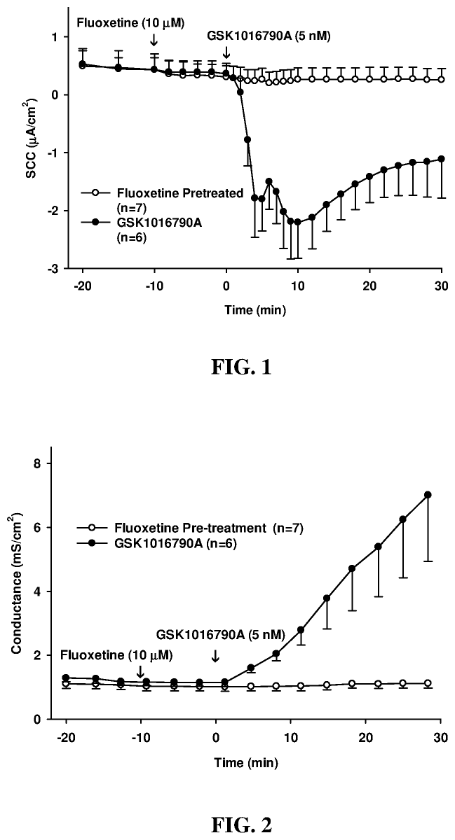

It has been found herein that in response to TRPV4 agonist administration, there is a transepithelial ion movement that is consistent with either anion absorption or cation secretion across the CP epithelium (see FIG. 1, black symbols). Furthermore, it has now been discovered that pretreatment with potassium channel inhibitors such as fluoxetine inhibits the TRPV4-stimulated ion transport (see FIG. 1, white symbols). This further suggests that the transepithelial transport is due to cation (specifically potassium) secretion into the CSF as fluoxetine blocks a certain type of potassium channels, notably calcium-activated potassium channels. Other blockers of calcium-activated potassium channels are also expected to be effective.

In another embodiment, it was found that norfluoxetine, an active metabolic product of fluoxetine, was effective at inhibiting the TRPV4-stimulated ion transport.

A particularly suitable dosage of potassium channel inhibitor typically depends on the specific form of hydrocephalus to be treated. More particularly, a suitable dosage of a potassium channel inhibitor used in the present disclosure will depend upon a number of factors including, for example, age and weight of an individual, at least one precise condition requiring treatment, severity of a condition, specific potassium channel inhibitor, nature of a formulation, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

Typically, treatment begins at the time of diagnosis of hydrocephalus, and is continued until the hydrocephalus and/or fluid build-up is resolved. In these situations, treatment can be restarted if hydrocephalus returns. In some embodiments, treatment begins shortly after an injury that can cause hydrocephalus occurs and can continue for a short period to prevent fluid build-up. The treatment time period in these embodiments can be determined by one skilled in the art such as, for example, a physician, a veterinarian, a scientist, and other medical and research professionals.

Any suitable method known to those skilled in the art can be used for administering the potassium channel inhibitor. Suitable methods for administering fluoxetine, for example, can be oral administration.

As used in the present disclosure, the potassium channel inhibitor can be administered as a pharmaceutical composition comprising potassium channel inhibitor (e.g., calcium-activiated potassium channel inhibitor such as fluoxetine) in combination with one or more pharmaceutically acceptable carriers. As used herein, the phrase “pharmaceutically acceptable” refers to those ligands, materials, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier”, as used herein, refers to a pharmaceutically acceptable material, formulation or vehicle, such as a liquid or solid filler, diluent, excipient, solvent or encapsulating material, involved in carrying or transporting the active compound from one organ or portion of the body, to another organ or portion of the body. Each carrier must be “acceptable” in the sense of being compatible with the other components of the composition (e.g., fluoxetine) and not injurious to the individual. Lyophilized compositions, which may be reconstituted and administered, are also within the scope of the present disclosure.

Pharmaceutically acceptable carriers may be, for example, excipients, vehicles, diluents, and combinations thereof. These compositions can be prepared by conventional means, and, if desired, the active compound (i.e., fluoxetine) may be mixed with any conventional additive, such as an excipient, a binder, a disintegrating agent, a lubricant, a corrigent, a solubilizing agent, a suspension aid, an emulsifying agent, a coating agent, or combinations thereof.

It should be understood that the pharmaceutical compositions of the present disclosure can further include additional known therapeutic agents, drugs, modifications of the synthetic compounds into prodrugs, and the like for alleviating, mediating, preventing, and treating the diseases, disorders, and conditions described herein.

The pharmaceutical compositions including the potassium channel inhibitors and pharmaceutical carriers used in the methods of the present disclosure can be administered to a subset of individuals in need as defined above. In particular, the individual in need is a human The individual in need can also be, for example, a research animal such as, for example, a non-human primate, a mouse, a rat, a rabbit, a cow, a pig, and other types of research animals known to those skilled in the art.

The disclosure will be more fully understood upon consideration of the following non-limiting Examples.

### EXAMPLES

### Example 1

In this Example, the effect of pre-administration of fluoxetine on TRPV4-stimulated ion transport response was analyzed.

In order to follow transepithelial ion movement, Ussing chamber electrophysiological techniques were used. The choroid plexus model is a high resistance, continuous cell line of porcine choroid plexus epithelial cells grown on permeable supports. Cultures to be used for electrophysiological analyses were grown in 6-well, Transwell filters for 10-12 days at which time the cultures developed a high transepithelial electrical resistance mimicking the in vivo situation. The cells were mounted on electrophysiological apparatuses for analysis of transepithelial ion flux in response to inhibitory or stimulatory reagents. Filters were excised, mounted in a Ussing chamber, and connected to a DVC-1000 Voltage/Current Clamp with voltage and current electrodes on either side of the membrane. Each half of the chamber contained a tapered fluid compartment with fittings for voltage electrodes (close to the epithelial membrane) and current electrodes (at the opposite end of the chamber). The fluid chamber was water jacketed to maintain a constant temperature (37° C.). The cells were bathed in serum-free media. Media were circulated in the chambers and oxygenated by means of a 5% CO₂/O₂ gas lift. The spontaneous transepithelial potential difference was measured and clamped to zero, and the resultant short-circuit (SCC) was monitored continuously as a measurement of net transepithelial ion flux. As per convention, a negative deflection in the SCC was either cation secretion (from blood to CSF) or anion absorption (CSF to blood). Transepithelial resistance (TER) was recorded every 200 seconds throughout each experiment by applying a 2 mV pulse and using the resulting deflection in the SCC to calculate the TER by Ohm's law. Conductances were calculated from the change in current during the voltages pulses. Specifically conductance is the change in current divided by the magnitude of the imposed voltage. In all cases, the graphs shown in each panel represent a series of control and experimental cultures that were grown and analyzed in parallel.

After the basal current was stabilized, effectors were added to one or both sides of polarized monolayer as dictated by the physiological situation.

When GSK1016790A, an agonist of TRPV4, was added to the PCP-R choroid plexus cell line (depicted by the black symbols) at time 0 minutes, there was a transepithelial ion transport response measured as short-circuit current (SCC) that was consistent with cation secretion into the CSF. When the cells were pre-treated with fluoxetine from time −10 to 0 minutes (depicted as white symbols), the GSK1016790A-stimulated (at 0 minutes) transepithelial potassium secretion in the direction of the CSF was completely inhibited (FIG. 1).

Further, conductance is a measurement of the permeability of the tissue to water and electrolytes. So, as the conductance goes up, one would expect greater ion and fluid permeability across the choroid plexus which is a barrier epithelium. The TRPV4 agonist causes a large increase in the conductance and pre-treatment with fluoxetine blocked this increased permeability (FIG. 2).

### Example 2

In this Example, the effect of pre-administration of fluoxetine or norfluoxetine on TRPVA-stimulated ion transport response was analyzed.

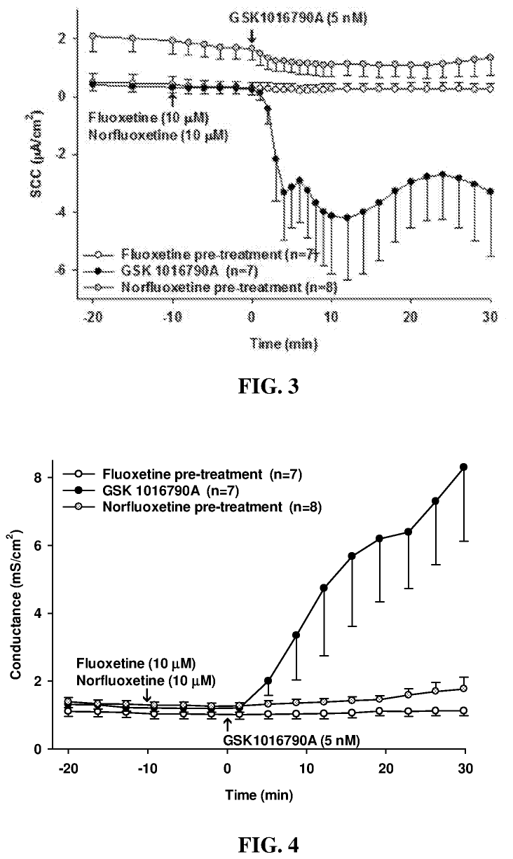

The same methods were used in this Example as in Example 1. As shown in FIG. 3, when the cells were pre-treated with fluoxetine or norfluoxetine from time −10 to 0 minutes (depicted as white symbols and grey symbols, respectively), the GSK1016790A-stimulated (at 0 minutes) transepithelial potassium secretion in the direction of the CSF was completely inhibited (FIG. 3). Further, pre-treatment with fluoxetine or norfluoxetine blocked this increased permeability (FIG. 4).

What is claimed is:

1. A method for inhibiting transepithelial ion transport in an individual in need thereof, the method comprising: administering a potassium channel inhibitor to the individual.

2. The method of claim 1 wherein the potassium channel inhibitor is selected from the group consisting of fluoxetine, norfluoxetine, and combinations thereof.

3. The method of claim 1, wherein the individual has a condition that results in the activation of a transient receptor potential cation channel subfamily V member 4 (TRPV4) agonist.

4. The method of claim 3, wherein the TRPV4 activation arises as a result of a condition selected from the group consisting of increased intracranial pressure, decreased cerebrospinal fluid (CSF) osmolarity, presence of inflammatory mediators that have escaped from the vasculature of the brain due to injury or inflammation.

5. The method of claim 1, wherein the potassium channel inhibitor is a calcium-activated potassium channel inhibitor and the individual is orally administered the calcium-activated potassium channel inhibitor.

6. A method for inhibiting hydrocephalic development in an individual in need thereof, the method comprising: administering a potassium channel inhibitor to the individual.

7. The method of claim 6 wherein the potassium channel inhibitor is selected from the group consisting of fluoxetine, norfluoxetine, and combinations thereof.

8. The method of claim 6, wherein the individual has a condition that results in the activation of a transient receptor potential cation channel subfamily V member 4 (TRPV4) agonist.

9. The method of claim 8, wherein the TRPV4 activation arises as a result of a condition selected from the group consisting of increased intracranial pressure, decreased cerebrospinal fluid (CSF) osmolarity, presence of inflammatory mediators that have escaped from the vasculature of the brain due to injury or inflammation.

10. The method of claim 6, wherein the potassium channel inhibitor is a calcium-activated potassium channel inhibitor and the individual is orally administered the calcium-activated potassium channel inhibitor.

11. A method for treating hydrocephalus in an individual in need thereof, the method comprising: administering a potassium channel inhibitor to the individual.

12. The method of claim 11 wherein the potassium channel inhibitor is selected from the group consisting of fluoxetine, norfluoxetine, and combinations thereof.

13. The method of claim 11, wherein the individual has a condition that results in the activation of a transient receptor potential cation channel subfamily V member 4 (TRPV4) agonist.

14. The method of claim 13, wherein the TRPV4 activation arises as a result of a condition selected from the group consisting of increased intracranial pressure, decreased cerebrospinal fluid (CSF) osmolarity, presence of inflammatory mediators that have escaped from the vasculature of the brain due to injury or inflammation.

15. The method of claim 11, wherein the potassium channel inhibitor is a calcium-activated potassium channel inhibitor and the individual is orally administered the potassium channel inhibitor.

16. A method for inhibiting fluid movement caused by transepithelial ion transport in an individual in need thereof, the method comprising: administering a potassium channel inhibitor to the individual.

17. The method of claim 16 wherein the potassium channel inhibitor is selected from the group consisting of fluoxetine, norfluoxetine, and combinations thereof.

18. The method of claim 16, wherein the individual has a condition that results in the activation of a transient receptor potential cation channel subfamily V member 4 (TRPV4) agonist.

19. The method of claim 18, wherein the TRPV4 activation arises as a result of a condition selected from the group consisting of increased intracranial pressure, decreased cerebrospinal fluid (CSF) osmolarity, presence of inflammatory mediators that have escaped from the vasculature of the brain due to injury or inflammation.

20. The method of claim 16, wherein the potassium channel inhibitor is a calcium-activated potassium channel inhibitor and the individual is orally administered the potassium channel inhibitor.
